SEQUENCE LISTING (21 sequences)

SEQ ID NO: 1 - DNA, 2282 bp, Artificial
  Human c-myc/Mouse Oestrogen Receptor Fusion c-mycERTam
gacgatgcccctcaacgttagcttcaccaacaggaactatgacctcgactacgactcggtgcagccgtatttctactgcgacgaggaggagaacttctaccagcagcagcagcagagcgagctgcagcccccggcgcccagcgaggatatctggaagaaattcgagctgctgcccaccccgcccctgtcccctagccgccgctccgggctctgctcgccctcctacgttgcggtcacacccttctcccttcggggagacaacgacggcggtggcgggagcttctccacggccgaccagctggagatggtgaccgagctgctgggaggagacatggtgaaccagagtttcatctgcgacccggacgacgagaccttcatcaaaaacatcatcatccaggactgtatgtggagcggcttctcggccgccgccaagctcgtctcagagaagctggcctcctaccaggctgcgcgcaaagacagcggcagcccgaaccccgcccgcggccacagcgtctgctccacctccagcttgtacctgcaggatctgagcgccgccgcctcagagtgcatcgacccctcggtggtcttcccctaccctctcaacgacagcagctcgcccaagtcctgcgcctcgcaagactccagcgccttctctccgtcctcggattctctgctctcctcgacggagtcctccccgcagggcagccccgagcccctggtgctccatgaggagacaccgcccaccaccagcagcgactctgaggaggaacaagaagatgaggaagaaatcgatgttgtttctgtggaaaagaggcaggctcctggcaaaaggtcagagtctggatcaccttctgctggaggccacagcaaacctcctcacagcccactggtcctcaagaggtgccacgtctccacacatcagcacaactacgcagcgcctccctccactcggaaggactatcctgctgccaagagggtcaagttggacagtgtcagagtcctgagacagatcagcaacaaccgaaaatgcaccagccccaggtcctcggacaccgaggagaatgtcaagaggcgaacacacaacgtcttggagcgccagaggaggaacgagctaaaacggagcttttttgccctgcgtgaccagatcccggagttggaaaacaatgaaaaggcccccaaggtagttatccttaaaaaagccacagcatacatcctgtccgtccaagcagaggagcaaaagctcatttctgaagaggacttgttgcggaaacgacgagaacagttgaaacacaaacttgaacagctacgggatccacgaaatgaaatgggtgcttcaggagacatgagggctgccaacctttggccaagccctcttgtgattaagcacactaagaagaatagccctgccttgtccttgacagctgaccagatggtcagtgccttgttggatgctgaaccgcccatgatctattctgaatatgatccttctagacccttcagtgaagcctcaatgatgggcttattgaccaacctagcagatagggagctggttcatatgatcaactgggcaaagagagtgccaggctttggggacttgaatctccatgatcaggtccaccttctcgagtgtgcctggctggagattctgatgattggtctcgtctggcgctccatggaacacccggggaagctcctgtttgctcctaacttgctcctggacaggaatcaaggtaaatgtgtggaaggcatggtggagatctttgacatgttgcttgctacgtcaagtcggttccgcatgatgaacctgcagggtgaagagtttgtgtgcctcaaatccatcattttgcttaattccggagtgtacacgtttctgtccagcaccttgaagtctctggaagagaaggaccacatccaccgtgtcctggacaagatcacagacactttgatccacctgatggccaaagctggcctgactctgcagcagcagcatcgccgcctagctcagctccttctcattctttcccatatccggcatatgagtaacaaacgcatggagcatctctacaacatgaaatgcaagaacgtggtacccctctatgacctgctcctggagatgttggatgcccaccgccttcatgccccagccagtcgcatgggagtgcccccagaggagcccagccagacccagctggccaccaccagctccacttcagcacattccttacaaacctactacatacccccggaagcagagggcttccccaacacgatctgagagctcc

SEQ ID NO: 2 - protein, 754 aa, Artificial
  Human c-myc/Mouse Oestrogen Receptor Fusion c-mycERTam
MPLNVSFTNRNYDLDYDSVQPYFYCDEEENFYQQQQQSELQPPAPSEDIWKKFELLPTPPLSPSRRSGLCSPSYVAVTPFSLRGDNDGGGGSFSTADQLEMVTELLGGDMVNQSFICDPDDETFIKNIIIQDCMWSGFSAAAKLVSEKLASYQAARKDSGSPNPARGHSVCSTSSLYLQDLSAAASECIDPSVVFPYPLNDSSSPKSCASQDSSAFSPSSDSLLSSTESSPQGSPEPLVLHEETPPTTSSDSEEEQEDEEEIDVVSVEKRQAPGKRSESGSPSAGGHSKPPHSPLVLKRCHVSTHQHNYAAPPSTRKDYPAAKRVKLDSVRVLRQISNNRKCTSPRSSDTEENVKRRTHNVLERQRRNELKRSFFALRDQIPELENNEKAPKVVILKKATAYILSVQAEEQKLISEEDLLRKRREQLKHKLEQLRDPRNEMGASGDMRAANLWPSPLIKHTKKNSPALSLTADQMVSALLDAEPPMIYSEYDPSRPFSEASMMGLLTNLADRELVHMINWAKRVPGFGDLNLHDQVHLLECAWLEILMIGLVWRSMEHPGKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHRRLAQLLLILSHIRHMSNKRMEHLYNMKCKNVVPYDLLLEMLDAHRLHAPASRMGVPPEEPSQTQLATTSSTSAHSLQTYYIPPEAEGFPNTI

SEQ ID NO: 3 - DNA, 20 bp, Artificial
  Primer Oligonucleotide
ctcctggcaaaaggtcagag

SEQ ID NO: 4 - DNA, 20 bp, Artificial
  Primer Oligonucleotide
aaggacaaggcagggctatt

SEQ ID NO: 5 - DNA, 20 bp, Artificial
  Primer Oligonucleotide
aaaggcccccaaggtagtta

SEQ ID NO: 6 - DNA, 20 bp, Artificial
  Primer Oligonucleotide
aaggacaaggcagggctatt

SEQ ID NO: 7 - DNA, 27 bp, Artificial
  Probe Oligonucleotide
acattcaagcagatctccatggcagca

SEQ ID NO: 8 - DNA, 26 bp, Artificial
  Primer Oligonucleotide
agtttgcagaagtttccaagttagtg

SEQ ID NO: 9 - DNA, 19 bp, Artificial
  Primer Oligonucleotide
aggtccgccctgtcatcag

SEQ ID NO: 10 - DNA, 24 bp, Artificial
  Probe Oligonucleotide
cggtgccatcccttgactcaacct

SEQ ID NO: 11 - DNA, 21 bp, Artificial
  Primer Oligonucleotide
tctggtgttctcaggcacaga

SEQ ID NO: 12 - DNA, 22 bp, Artificial
  Primer Oligonucleotide
caaccagaaaaacccgttgttc

SEQ ID NO: 13 - DNA, 29 bp, Artificial
  Probe Oligonucleotide
ccgtaagtggagcctgatttccctaagga

SEQ ID NO: 14 - DNA, 22 bp, Artificial
  Primer Oligonucleotide
aaaggctgagtcaagggatgag

SEQ ID NO: 15 - DNA, 25 bp, Artificial
  Primer Oligonucleotide
agctttcttaaagagcaaaccagaa

SEQ ID NO: 16 - DNA, 25 bp, Artificial
  Probe Oligonucleotide
accacagtccatgccatcactgcca

SEQ ID NO: 17 - DNA, 23 bp, Artificial
  Primer Oligonucleotide
caaggtcatccatgacaactttg

SEQ ID NO: 18 - DNA, 20 bp, Artificial
  Primer Oligonucleotide
gggccatccacagtcttctg

SEQ ID NO: 19 - DNA, 26 bp, Artificial
  Probe Oligonucleotide
catcactatcggcaatgagcggttcc

SEQ ID NO: 20 - DNA, 19 bp, Artificial
  Primer Oligonucleotide
gagctatgagctgcctgac

SEQ ID NO: 21 - DNA, 21 bp, Artificial
  Primer Oligonucleotide
agtttcatggatgccacagga

(19) United States
(12) Patent Application Publication
(10) Pub. No.: US 2008/0131404 A1
(43) Pub. Date: Jun. 5, 2008

(54) Hepatocytes

(76) Inventors: Deborah Johnson, Surrey (GB); John Sinden, Surrey (GB); Peter Goldfarb, Surrey (GB); Anders Svanberg, Surrey (GB)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 11/574,437

(22) PCT Filed: Sep. 21, 2005
(86) PCT No.: PCT/GB05/03631

§ 371 Date: Oct. 18, 2007

(30) Foreign Application Priority Data
Sep. 21, 2004 (GB) 0420963.1

(51) Int. Cl. C12N 5/08; C12N 5/06; C12N 5/00; C12Q 1/02; A61K 35/407; A61P 1/16

(52) U.S. Cl. 424/93.7; 435/325; 435/370; 435/375; 435/29

(57) ABSTRACT

The invention relates to the production of hepatocyte cell lines useful in toxicology screens or therapy. A mammalian hepatocyte comprises, as a single polypeptide, a fusion protein comprising a c-myc protein and an oestrogen receptor, or functional fragments thereof.

### FIELD OF THE INVENTION

This invention relates to conditionally-immortalised hepatocyte cells that can be scaled up for clinical and commercial application.

### BACKGROUND TO THE INVENTION

The liver is responsible for the detoxification of drugs and poisons from the body. The most common cell type in the liver are hepatocytes; each hepatocyte has the capacity to perform each task required of the whole organ. Due to the metabolism of drugs in the liver, Absorption, Distribution, Metabolism, Excretion and Toxicity (ADME/Tox) testing of drug candidates is performed on liver tissue to assess potential toxicity in vivo.

Traditionally ADME/Tox tests have been performed late in the process of developing new drugs, but the number of new drugs that fail because of toxicity is about 30%. There is therefore great interest in the development of high-throughput ADME/Tox screening that can be applied early in the drug development process, thereby saving time and money.

Many in vitro ADME/Tox studies focus on the liver's main metabolic enzymes, the cytochrome P450 enzyme family, although studies on P-glycoprotein and absorption studies with the CaCo-2 cell line are also commonly used.

Several biological assay systems are used to evaluate cytochrome P450 enzyme isoforms, but they all have some major disadvantages in their ability to predict in vivo toxicity. The main disadvantage of current assays is that only a limited number of liver enzymes can be studied at once, limiting the similarity between the assay and the situation in the liver itself^{1,2}.

Human hepatocytes are the closest in vitro model of the human liver. Primary hepatocyte cultures are often used in ADME/Tox studies, as these express a “normal” hepatocyte phenotype. However, the availability of these cells is low and the expression of markers varies between batches. Foetal hepatocytes proliferate readily when dissociated and plated in cell culture under the right culture conditions, but the expression levels of many mature enzymes differ in foetal hepatocytes. For example, the level of cyp3A4, one of the main metabolic enzymes, is much lower in foetal than mature hepatocytes³. Adult hepatocytes also prove problematic in culture. With a low proliferation capacity, it is difficult to induce them to proliferate more then once in vitro, even with stimulation by growth factors⁴.

The problems associated with primary hepatocytes led to the development of hepatocyte cell lines. Several human hepatocyte cell lines are currently available, some derived from adult liver and some from foetal liver, either immortalized with virus or simply cultured without immortalisation^{5,6}. However, no cell line expresses a “normal” human hepatocyte phenotype, mainly because the expression of many hepatocyte markers, including cytochrome P450, decreases rapidly or completely disappears in cell culture⁷. There are seven classes of hepatocyte mRNA markers, as indicated in table 1, together with examples in each class.

TABLE 1
Liver mRNA markers for phenotype assessment

Transcription factors:
Hepatic nuclear factor 1 (HNF1)
Hepatic nuclear factor 2 a, b
(HNF2 a, b)
Hepatic nuclear factor (HNF4)
Albumin D-element binding protein
(DBP)
Liver activator protein (LAP)
Pregnane X receptor (PXR)
Pregnane X receptor a (PXR a)
Constitutively active receptor also
known as constitutive androstane
receptor (CAR)
Glucocorticoid receptor (GR)
Peroxisome Proliferator Activated
Receptor a (PPAR a)
Metabolic enzymes:
Urea cycle enzymes
Arginine synthesis enzymes
Serine aminotransferase
phosphoenolpyruvate carboxykinase
(PEPCK)
Tyrosine oxidase
Fatty acyl-CoA oxidase (FACO)
Peroxisomal enoyl-CoA hydratase/3-
hydroxyacyl CoA dehydrogenase
“bifunctional enzyme” (BE)
Hormone-sensitive lipase (HSLIP)
Camitine palmitoyltransferase 2 (CPT-2)
Medium chain acyl-CoA dehydrogenase
(MCACD)
3-ketoacyl-CoA thiolase (KCAT)
Lecithin:cholesterol acyltransferase
(LCAT)
Delta-aminolevulinate synthase (DALS)
Liver specific proteins/enzymes:
Albumin
Alpha fetoprotein (AFP)
Transferrin
Fibrinogen
Factor X
Plasmin
Selenium-dependent glutathione
peroxidase-1 (GPX-1)
Extracellular superoxide dismutase
(SODxc)
Apolipoprotein B
Drug metabolising enzymes:
Cytochrome P-450 isoenzymes (CYPs)
Cytosolic epoxide hydrolase (Ehc)
Mitochondrial epoxide hydrolase (Ehm)
Glutathione sulfotransferases (GSTs)
UDP-glucuronosyltransferases (UGTs)
Phenolsulphotransferases (SULTs)
N-acetyltransferases (NATs)
Estradiol sulfotransferase (EST)
Phenol/aryl sulfotransferase (PST)
Hydroxysteroid sulfotransferase (HSST)
Cell cycle & apoptosis:
Apoptotic protease-activating factor 1
(apaf1)
Bcl-x
Caspase3
Cyclin-dependent kinase 1 (cdc2)
CyclinD1
daxx also known as death associated
protein-6 (DAP-6)
mdm-2
Heat shock protein 70 (HSP 70)
Receptors:
Hepatocyte growth factor receptor
(HGFR)
Insulin-like growth factor-1 receptor
(IGF-1 Rec)
Epidermal growth factor receptor
(EGFR)
Transforming growth factor b receptor
(TGFb-R)
Prostacyclin or prostaglandin I 2
receptor (PRCR)
Transporters:
P-glycoprotein (PGPs)
Na-taurocholate cotransporting
polypeptide (NTCP)
Canalicular multispecific organic anion
transporter (CMOAT)

It is possible to induce and upregulate the expression of some liver markers by adding substances such as 3-methylcholantrene and oncostatin M^{8,9} to the media. However, it is not currently possible to upregulate the levels of all or most hepatocyte enzymes to a “normal” level by adding substrates to the media or altering culture conditions¹⁰. Several hepatocyte lines are available from other species, including rats and primates^{11,12}, but these also have the problem of low expression of markers and the additional problem of interspecies differences.

There is therefore the need for a human hepatocyte cell line expressing normal liver functions and markers. Such a cell line, that could be used to study most phase I and II drug metabolism enzymes including the study of cell-cell contact, would be an unlimited cell source, could be used in a high throughput system, give highly reproducible data, have no inter-species differences, and would be cost effective¹.

### SUMMARY OF THE INVENTION

The present invention is based upon the construction of a conditionally immortalised hepatocyte that is immortal when 4-hydroxytamoxifen (4-OHT) is present in the cell culture but which express normal hepatocyte markers when 4-OHT is not present. A c-myc/oestrogen receptor fusion is responsible for conferring conditionally immortal character to the hepatoctye.

According to a first aspect of the invention, a mammalian hepatocyte comprises a fusion protein comprising an oncoprotein of the myc family and an oestrogen receptor, or functional fragments thereof, expressed as a single polypeptide chain.

According to a second aspect of the invention, a method of conditionally immortalising a hepatocyte comprises the steps of:

(i) expressing in the hepatocyte a fusion protein comprising an oncoprotein of the myc family and an oestrogen receptor, or functional fragments thereof, expressed as a single polypeptide chain; and

(ii) contacting the hepatocyte formed by step (i) with a ligand of the oestrogen receptor, thereby conditionally immortalising the hepatocyte.

The hepatocytes according to the invention are particularly useful for in vitro testing of potential drugs, especially in ADME/Tox testing.

According to a third aspect of the invention, a method of evaluating the suitability of a compound for use as a drug, in vitro, comprises the steps of:

(i) contacting a hepatocyte comprising a fusion protein comprising an oncoprotein of the myc family and an oestrogen receptor, or functional fragments thereof, expressed as a single polypeptide chain, with the potential drug compound;

(ii) measuring the response of the hepatocyte, to thereby determine the effect of the compound on the hepatocyte and evaluate the suitability of the compound for use as a drug. Preferably, the heptocytes will be growth-arrested by the removal of the oestrogen receptor ligand, eg 4-hydroxytamoxifen (4-OHT) from the culture medium, and that the hepatocytes will develop a fully differentiated phenotype.

Further aspects of the invention are the use of the hepatocytes as a medicament, and in the manufacture of a medicament for the treatment of liver disease. Yet another aspect of the invention is the use of a hepatocyte according to the invention in a Liver Assisting Device (LAD).

### DETAILED DESCRIPTION OF THE INVENTION

The present invention identifies that expression of a myc/oestrogen-receptor fusion protein in an hepatocyte conditionally immortalises the hepatocyte. When the hepatocyte is cultured in the presence of a ligand of the oestrogen receptor, it is immortal. The removal of the ligand from the hepatocyte removes the immortality of the cell, which then displays the “normal” characteristics and markers of a hepatocyte, as would be expected from a hepatocyte in situ in a liver.

As used herein, the term “hepatocyte” refers to any epithelial cell obtainable from a liver that is capable of performing one or more functions carried out by the liver. Hepatocytes from any species are within the scope of the invention, although it is preferred that the hepatocyte is mammalian, most preferably human. Foetal or adult hepatocytes may be used, as can hepatocytes that have differentiated from a precursor cell in vitro, e.g. hepatocytes differentiated from embryonic stem cells or pluripotent stem cells (see U.S. Pat. No. 6,506,574).

Hepatocytes according to the present invention maintain markers characteristic of “normal” hepatocytes. There are seven classes of hepatocyte mRNA markers, as indicated in Table 1, together with examples of each class. In the investigation detailing the current patent application, five of these markers were used in the characterization of the human foetal liver clones, namely albumin, α-fetoprotein, cytokeratin-18, cyp3A4 and cyp3A7.

As used herein, the term “immortal” refers to a cell with the ability to undergo extended proliferation. The hepatocytes of the current invention are “immortal” when grown in the presence of a ligand of the oestrogen receptor, eg 4-OHT. A culture of cells in vitro expanded from a single cell or a colony of cells is referred to as a “cell line”, as will be appreciated by one skilled in the art. This is in contrast to primary cells which can only divide a limited number of times, normally less than 10-20 divisions, before senescence is reached and the cell eventually dies. As used herein, the term “conditionally immortal” refers to a cell that is dividing and immature under certain, specific, growth conditions but which is a fully mature cell under other conditions. According to the current invention, the environmental condition responsible for immortalising the cells is the presence of the oestrogen receptor ligand.

The feature of the hepatocytes that allows them to be continually immortal, and therefore be immortalised by the oestrogen receptor ligand, is the presence of a myc/oestrogen receptor fusion protein. Without wishing to be bound by theory, it appears that the ligand (eg 4-OHT) activates the oestrogen-receptor. Activation of the oestrogen-receptor allows the oncoprotein myc to dimerise and be transported into the nucleus where it acts as a transcription factor, initiating expression of genes allowing proliferation to occur. Without the ligand, the fusion protein comprising the myc oncoprotein is still expressed but it remains in the cytoplasm and no further proliferation occurs. The ligand can therefore be added to the media to make the hepatocytes proliferate (immortally), and can be withdrawn allowing the cells to behave like normal non-proliferating hepatocytes.

The hepatocytes of the invention are conditionally immortal due to the expression of a myc/oestrogen-receptor fusion protein. As used herein, the term “fusion protein” refers to a recombinant protein that comprises two protein or peptide sequences, that are naturally expressed separately, expressed as a single polypeptide chain.

The fusion protein may comprise any myc protein and any oestrogen-receptor, or any fragments of these proteins that maintain the ability to be activated by the oestrogen receptor ligand and activate transcription leading to cell proliferation, respectively. Preferably, the myc protein is c-myc. Preferably, the oestrogen-receptor has a mutation that prevents high affinity binding to 17β-oestradiol, without affecting the high-affinity binding to the ligand 4-OHT. This mutation may be a deletion, substitution or addition of one or a number of amino acid residues. In a preferred embodiment, the fusion protein consists of a human c-myc gene fused to a mouse oestrogen receptor. More preferably, the fusion protein comprises the amino acid sequence identified herein as SEQ ID No. 2. As stated previously, any homologue or functional fragment of SEQ ID No. 2 is within the scope of the invention.

As used herein, the term “homologue” refers to the similarity or identity between two or more biological polymers, including DNA, RNA and protein sequences. The concept of sequence identity is well known in the art, and refers to the level of identity between two sequences. Equally well known is the concept of similarity, wherein conservative differences between two sequences, which do not have a large effect on structure or function, are included when considering the likeness between two sequences. For example, a glutamic acid may be substituted for an aspartic acid without a large effect on the protein structure or function, these residues are “similar”. In contrast, an aspartic acid residue shows no similarity to a phenylalanine residue. Homologues included within the scope of the invention must have a high similarity to the mouse oestrogen-receptor and human c-myc sequences identified herein. Identity and similarity may be calculated using any well known algorithm, for example Needleman-Wunsch, Smith-Waterman, BLAST or FASTA. Homology may be determined at the nucleic acid or amino acid level. Preferably, homologues within the scope of the invention have at least 50%, more preferably at least 60%, even more preferably greater than 70% and most preferably greater than 80%, for example 90% homology at the amino acid or nucleic acid level as calculated using the BLAST programme under default conditions.

The hepatocytes of the invention express the c-myc/oestrogen receptor fusion protein. The polynucleotide molecule encoding the fusion protein, referred to herein as the “fusion polynucleotide”, may be present in the hepatocyte in any form, for example as a plasmid within the hepatocyte or integrated into the host's genome.

It is preferred that the hepatocytes are conditionally immortalised by incorporation of the fusion polynucleotide into the hepatocyte genome. Methods for the integration of heterologous polynucleotides into a host genome are well known in the art and any suitable method may be used. Preferably, retroviral infection is used to integrate the fusion polynucleotide into the hepatocyte genome. Retroviral vectors for the integration of genetic material into foreign genomes are well known in the art, and any may be used. Preferably, the vector is an amphotropic retrovirus, most preferably, the vector is pLNCX (BD Biosciences Clontech).

The vector is packaged together with the fusion polynucleotide in any suitable virus producing cell. The virus is preferably produced by Fly-CO42 cells originating from the TEFLY virus producer cell line.

The pLNCX vector comprises a LTR promoter which drives a neomycin resistance gene. Neomycin (also known as geneticin and G418) is used in the media during selection so that only cells expressing the neomycin resistant gene survive. A titration of neomycin can be performed on non-infected target cells to establish the concentration required to eliminate uninfected cells. Any antibiotic resistance gene may be used in order to aid selection of infected cells, although antibiotic resistance is not essential.

Any promoter can be used to promote expression of the fusion polynucleotide. Preferably, this is a different promoter to that used to promote expression of any antibiotic resistance gene. Preferably, a cmv promoter drives expression of the fusion polynucleotide.

An example of a suitable fusion polynucleotide is c-mycERTam, identified herein as SEQ ID No. 1, comprising a human c-myc gene fused to a mouse oestrogen receptor that is mutated to remove high affinity binding to 17β-oestradiol without affecting the high affinity binding to the synthetic drug 4-OHT. Any mutation to the polynucleotide that causes this functional change in the protein is within the scope of the invention, including an addition, substitution or deletion. Preferably, the mutation is a point mutation. In the preferred embodiment, a point mutation is introduced to alter the wild-type glycine at amino acid position 681 to arginine. Homologues and functional fragments of c-mycERTam are within the scope of the invention.

It will be apparent to one skilled in the art in order for the retroviral vector to integrate into the hepatocyte genome, the hepatocyte needs to be proliferating. To maximise integration, the virus should be added when the hepatocytes are proliferating at a high rate¹³. To investigate proliferation rate it is preferable to stain for the proliferation marker Ki67, a nuclear protein expressed in late G1, S, M and G2 phases of the cell cycle but not in G0¹⁴. To further increase the success rate for the infection, a facilitator like hexadimethrine bromide (also known as polybrene) may be added to the media during infection. This can be toxic to some cells at high concentrations, but it has been successfully used for infection of human foetal hepatocytes^{15,17}.

When studying hepatocytes according to the invention, it is preferred that all 4-OHT is removed prior to any study, as 4-OHT can cause inhibitory effects on the cytochrome p450 enzyme CYP3A4, and may also react with other chemicals.

Applications of the hepatocytes according to the invention include any method involving in vitro assessment of the effect of a chemical, for example a drug or poison, upon a liver cell, including but not limited to the study of endobiotic and xenobiotic metabolism, hepatotoxicity and hepatocarcinogenicity, liver-associated human restricted pathogens such as hepatitis and malaria, and cardiovascular research.

The cells are particularly useful in ADME/Tox testing, to determine whether a drug candidate is suitable for use in vivo, in particular to assess its effects upon hepatocytes (and therefore the liver) and determine toxicity. There are many techniques used in ADME/Tox testing, as will be appreciated by one skilled in the art. These techniques generally involve contacting hepatocytes in vitro with a potential drug compound and measuring the response of the hepatocytes. The response may be measured in a variety of ways, including but not limited to the measurement of growth and proliferation rates, expression profiles and enzyme activity assays. A titration of the potential drug compound may be carried out, to assess the point, if any, at which the compound becomes toxic to the hepatocytes. Based upon the data of such tests, the suitability of the compound for use as a drug is determined.

In a preferred embodiment, culture conditions will optimise the long-term expression of fully differentiated metabolic expression markers. Such culture methods are known in the art and include 3-dimensional cultured aggregates, collagen sandwich cultures, as described for example in Richert et al., (2002)²⁸.

In particular, it is envisaged that the cells may be used in high throughput screening.

Suitable high throughput screening assays are disclosed in Waring et al., (2001)²¹, Barton et al., (2002)²², Hamadeh et al., (2002)²³, de Longueville et al., (2003)²⁴, Adam et al., (2001)²⁵, Seow et al., (2001)²⁶ and Anderson (1991)²⁷, the content of each being incorporated herein by reference.

An advantage of using 4-OHT as the proliferation signal in hepatocytes according to the invention is that this synthetic chemical is not normally present in humans, allowing the hepatocytes according to the invention to be used as a medicament. In particular, the hepatocytes can be used in therapeutic cell lines¹⁸, for use in transplantation therapy. It is recognised that transplantation of hepatocytes is an option to treat diseases of the liver, where transplantation of healthy hepatocytes into a diseased or damaged liver can replace cells damaged by disease. Cell transplantation is seen as a preferable alternative to organ transplantation. Any disease that impairs liver function may be treated by the transplant of hepatocytes according to the invention, including but not limited to cancer, cirrhosis, all forms of hepatitis and damage caused by drug or alcohol abuse. Veterinary treatments involving the hepatocytes is also within the scope of the invention.

The hepatocytes according to the invention may also be used in a Liver Assisting Device (LAD), also known as a Bioartificial liver device. An LAD is a device capable of performing liver function, for example in the case of acute liver failure. LADs may be extracorporeal, wherein they perform a dialysis role, or be implanted into the patient. The primary use of LADs is to support a patient in the period immediately following an acute liver failure, but may also be used after transplantation to support the patient until the transplanted liver is fully functional. LADs commonly comprise at least one chamber filled with metabolically-active hepatocytes. Then patients blood is contacted with the hepatocytes, which process the blood as a liver would.

The invention is further illustrated by the following non-limiting examples.

### EXAMPLE 1

### Materials and Methods

All chemicals were purchased from Sigma unless otherwise mentioned.

### Tissue Culture

All cells were grown at 37° C. in 5% CO₂ in millennium incubators (Jencon). The cells were fed on a regular basis with media change three times a week. The cells were passaged when at 90% confluency.

### Media

Eight types of media were used during tissue culture. Human foetal liver cells and clones were cultured in all of these medias. The medias were sterile filtered with 1 L filter units (Nalgene) and pre warmed to 37° C. before use. The medias were called media: A, B, C, D, E, F, G and H and consisted of the following:
Media A: Arginine free Williams E standard medium (Gibco), Sodium bicarbonate, 2.2 g/L, dialysed fetal bovine serum (dFBS) (Gibco 26400-044), 10%, L-glutamine, 2 mM, insulin, 100 nM, penicillin-streptomycin (Gibco), 100 IU/ml, L-ornithine, 0.4 mM, hydrocortisone, 5.5 μM, epidermal growth factor (EGF), 20 ng/ml.
Media B: Dulbecco's modified eagle's medium (DMEM) with foetal bovine serum (FBS) (HyClone), 10%, gentamicin, 50 mg/ml, L-glutamin, 2 mM, EGF, 20 ng/ml
Media C: Williams E standard media (Gibco): DMEM (1:1), B27 supplement mix (50×) without vitamin A (Gibco), 1:50, FBS, 10%, L-glutamine, 2 mM, penicillin-streptomycin, 100 IU/ml, hydrocortisone, 5.5 μM, insulin, 100 nM, nicotinamide, 10 mM, EGF 20 ng/ml, hepatocyte growth factors (HGF) (Peprotech),10 ng/ml.
Media D: Williams E standard media (Gibco): DMEM (1:1), FBS, 10%, L-glutamine, 2 mM, penicillin-streptomycin, 100 IU/ml.
Media E: Williams E standard media (Gibco): DMEM (1:1), FBS, 4%, L-glutamine, 2 mM, penicillin-streptomycin, 100 IU/ml.
Media F: DMEM F12, human serum albumin 0.03%, transferrin, human 100 mg/ml, putrescine dihydrochloride 16.2 mg/ml, insulin, human recombinant 5 mg/ml, progesterone 60 ng/ml, L-glutamine 2 mM, sodium selenite (selenium) 40 ng/ml, heparin, sodium salt 10 units/ml, corticosterone 40 ng/ml, gentamycin 50 mg/ml, nicotinamide, 10 mM, EGF 20 ng/ml, HGF, 10 ng/ml.
Media G: Williams E standard media (Gibco): DMEM (1:1), B27 supplement mix (50×) without vitamin A (Gibco), 1:50, FBS, 10%, L-glutamine, 2mM, penicillin-streptomycin, 100 IU/ml, hydrocortisone, 5.5 μM, insulin, 100 nM.
Media H: DMEM, FBS, 10%, insulin 100 nM, hydrocortisone, 5.5 μM, penicillin-streptomycin, 100 IU/ml.

### Coating

Both coated and uncoated tissue culture plasticware were used. The coating was done with a solution of collagen type 1, calfskin, diluted in sterile bottled water (Fresenius Kabi) and added in a concentration of 6 μg/cm². The plastic ware was finally washed in PBS before use.

### Liver

Human foetal livers were obtained following terminated pregnancies. Local NHS ethical committee approval had been obtained prior to the research being started.

### Dissociation

The human foetal liver was received on wet ice in RPMI media after shipment. The liver was washed 2 times in +4° C. calcium free HEPES pH 7.2 (Gibco) and once in +4° C. calcium free HEPES pH 7.2 with ethylene glycol-bis(b-aminoethylether)-N,N,N′,N′-tetraacetic acid (EGTA), 0.5 mM. The liver was minced with scalpels and put into a 37° C. enzyme solution for 20 minutes consisting of calcium-free hanks balanced salt solution (HBSS) containing collagenase A (Roche), 0.05%, DNase I (Roche), 0.005%, hylaronidase, 0.0125% and dispase II (Roche), 0.025% supplemented with 5 mM calcium chloride. The dissociated liver was filtered through a sterile nylon mesh and collected in +4° C. media B. To remove some of the small blood cells and other cells the cells were spun 3 times at 50 g and the supernatant discarded each time. A cell and viability count was performed with a haemocytometer. Some cells were purified further with immunomagnetic isolation. The cells were plated on since earlier collagen coated tissue culture plastic ware in media B and left to attach for 4 hours. The cells were then washed once with 37° C. phosphate buffered saline pH 7.4 without calcium chloride and magnesium chloride (PBS) (Gibco) to remove debris and unattached cells, and media A was added.

### Immunomagnetic Isolation

The immunomagnetic isolation was done as a positive selection for hepatocytes and hepatoblasts from the dissociated liver and as a further purification step for removal of nonepithelia cells. Approximately 10⁷ cells from the dissociated liver were added to 450 μl PBS and 50 μl anti-HEA-125 (human epithelial antigen) antibodies, mouse monoclonal IgG1 (Progen 61004) and incubated at 37° C. for 30 minutes in a falcon tube. The anti-HEA-125 antibodies recognise an epithelial cell surface glycoprotein and the antibody label most epithelial cell types including hepatocytes and hepatoblasts but seem to not label nonepithelial tissue¹⁹. The tube was then spun at 500 g for 2 minutes to pellet the cells and the supernatant discarded to remove unbound antibodies. The cells were resuspended in 500 μl +4° C. PBS and 10 μl dynabeads M-450 (Dynal), which are coated with sheep anti-mouse IgG antibodies, and incubated at +4° C. for 30 minutes so the beads could attach to cells labelled with anti-HEA-125 antibodies. 4.5 ml PBS was added to the falcon tube and it was placed in a magnet (polyATtract system 1000, Promega) for 3 minutes at +4° C. The supernatant was carefully discarded to remove cells not attached to a magnetic bead. This step was repeated 3 times. The cells were finally resuspended in media B and plated on collagen precoated tissue culture plastic ware.

### Passaging

Each time the cells were passaged or removed from a flask the flask was washed once with 37° C. PBS. After that a 37° C. 1× trypsin-versene mix (Bio Whittaker) was added for 10 minutes and the cells detached from the plastic. An equal volume of media was added to the trypsin-versene mix to neutralise the trypsine and the cells were spun for 5 minutes at 500 g in a falcon tubes. The supernatant was aspirated and the cells resuspended in 1 ml of media and a cell count was preformed with a haemocytometer. The cells were passaged in a ratio 1:3 into a new flask.

Freezing/thawing

When freezing the cells the cell pellet was resuspended in 900 μl of media and 100 μl of cryosure-dimethyl sulfoxide (DMSO) (Quest biomedical) in a cryo-safe vial (Nunc). The cells were put into −80° C. for 24 hours in a “Mister Frosty” cryopreservation freezing container (Nalgene), where the temperature decreased at approximately 5° C./minutes. After 24 hours the cells were moved to liquid nitrogen for long-term storage. When thawed the cells were removed from the liquid nitrogen and placed in a 37° C. water bath until thawed and then mixed with 10 ml of 37° C. media and spun for 500 g for 5 minutes. The supernatant was discarded and the pellet resuspended in fresh media by gentle pipetting and plated in a new flask.

Harvesting of Virus

Virus producer cells from clone Fly-C042 were cultured to confluence in several T-175 flasks. The media used was DMEM glutamax (Gibco) supplemented with FBS, 10%, L-glutamin, 2 mM, and penicillin-streptomycin, 100 IU/ml. When confluent the flasks were washed 3× with PBS, and media A was added to the virus producing cells for 8 hours, 18 ml/T175 flask. The media was harvested and filtered through a 0.45 μm filter (Sartorius) and aliquoted, 5 ml media/falcon tube, and snap frozen in liquid nitrogen and stored in −80° C.

Infection

3 days after the dissociation of the human foetal liver the media was aspirated and the cells washed once with PBS and exposed to thawed media containing the virus and hexadimethrine bromide, 8 μg/ml, for 8 hours. After 8 hours of infection ordinary media was added and the infection was repeated the next day. The infection was done in 10 cm petri dishes with cell concentrations ranging from 200cells/dish to a 100% confluent dish. The cells were passaged after the second round of infection into different medias, and left for 2 days before selection started. After the infection 4-OHT, 100 nM, was always added to the media.

### Selection

To determine what concentration of neomycin to use on the liver cells exposed to the virus, in order to eliminate all or the majority of non-infected cells, but not the infected cells, a titre of neomycin was set up. Human foetal liver cells from a dissociated liver that had never been exposed to the immortalising virus were plated in 6-well plates and exposed to neomycin concentrations ranging from 0-900 μg/ml for 12 days in triplicate. 100, 500 and 2000 cells/well were plate and the cells were left for 2 days before the neomycin was added. The growth and proliferation of the cells were studied visually with the TMS-F microscope (Nikon). Based on the result from the neomycin titre a concentration of 250 μg/ml neomycin was used for 2 weeks on the liver cells exposed to the immortalising virus.

### Immunocytochemistry

For early assessment of the cells, immunocytochemistry was used. It is not possible to quantify what the expression level is but the benefit with immunocytochemistry is that few cells are needed and it is possible to see how many cells express a specific marker in a cell population and it is also possible to see if some cells have higher expression then others. This can be of great value in the process of choosing cells and cell lines to go forward with and which cells/cell lines to discard.

The antialbumin antibody had no cross-reactivity with bovine albumin according to the specification sheet from Sigma, cross-reactivity could have cause unspecific binding to FBS in the culture media. The antifibroblast antibody reacted with the b-subunit of prolyl-4-hydroxylase and with disulphideisomerase. The 2 hepatoma cell lines HepG2 and HuH7 were used for the optimisation of the staining and as positive controls during the experiments for hepatocyte markers and the human brain cell line, CTX-08, were used as negative control. All staining were performed in multiwell format and the following procedures were used. The media was aspirated and the cells were washed once with PBS and fixed in 4% paraformaldehyde in PBS for 15 minutes at room temperature (RT). The 4% paraformaldehyde was aspirated and the cells washed 3 times with PBS. For ck-18, fibroblast and Ki67 staining the cells were permubelised for 20 minutes in 0.1% triton x-100 in PBS. For albumin and AFP staining the cells were permubelased with ice-cold methanol for 5 minutes. The cells were then washed once with PBS and blocked, 30 minutes, in 10% normal goat serum (NGS) (Vector) in PBS. Primary antibodies were added in 1% NGS in PBS for 1 hour at RT except for Ki67, which was added for 2 hours. The wells were washed 3×5 minutes with PBS and the secondary antibody were added in PBS for 1 hour at RT. The secondary antibody excited at a wavelength of ˜488 nm and gave emission at ˜525 nm (green emission). After 1 hour's incubation the wells were washed 3×5 minutes in PBS and hoechst, a chemical that stains DNA was added for 2 minutes at RT, 1:25 000 in PBS. Hoechst excited at ˜350 nm and gave emission at ˜425 nm (blue emission). Finally the wells were washed 3×5 minutes in PBS and left in the last wash. For the anti c-myc staining the cells were fixed, blocked and permubelised in triton x-100 as described earlier. The primary antibody was then left O.N. the secondary antibody was left for 1.5 hours. All analysing was done with the fluorescence microscope Leica DMRA and the digital camera C4742-95 (Hamamatsu) and computer software Hamamatsu image PRO.

### Total RNA Extraction

For the total RNA extraction the RNeasy mini kit (Qiagen 74104) was used. A cell pellet to up to 5×10⁶ cells was used per extraction. The cells were disrupted with 350 μl RLT buffer with 2 mercaptoethanol, 10 μl/ml, and the sample homogenized by passed 10 times through a 20-gauge cyringe. Absolute alcohol (Joseph Mills Ltd) was diluted to 70% and 350 μl added to the sample and mixed by pipetting. The sample was transferred to an RNeasy mini column and spun at 8000 g for 15 s. 350 μl RW1 wash buffer was added and spun at 8000g for 15s. 10 μl DNase 1 RNase-free (Qiagen 79254) in 70 μl RDD buffer (Qiagen) was added to the RNeasy mini column membrane for 15 minutes. 350 μl RWI wash buffer was added and spun at 8000 g for 15s, 500 μl RPE buffer was added twice and the sample were spun for 15 seconds and 2 minutes at 8000 g each time. Finally 50 μl RNase-free water was added to the column and spun at 8000 g for 1 minute. The 50 μl water containing the RNA was collected in a Rnase free tube and purity and quantity was checked with the spectrofotometer GeneQuant pro (Amersham pharmaceutical biotech AB). The sample was stored in −80° C.

### Making cDNA

To make cDNA, extracted total RNA was thawed on ice and 50 ng was mixed with the following in DNase/RNase free tubes on ice: RNasin RNase inhibitor 10.000 u, (Promega N2115), 0.25 μl, random primers 3 μg/pl (Invitrogen 48190-011), 0.067 μl and buffer RT 10×, 2 μl, dNTP-mix 5 mM each, 2 μl, Sensiscript RT, 1 μl, and RNase-free water (all from Quiagen kit 205213) were mixed to a final volume of 20 μl. The tubes were placed in a 37° C. water bath for 60 minutes, transferred to a 93° C. heat block for 5 minutes and rapidly cooled on ice. The cDNA was stored in a −20° C. freezer.

### PCR for c-myc-ER

For the PCR of the c-myc-ER the titanium taq PCR kit (Clontech laboratories) was used, see table 2 for the primers used. The PCR reaction conditions were standard. The PCR machine used was GeneAmp PCR system 2700. For analysis, 15 μl of the PCR product was mixed with 5 μl 6× loading dye solution (MBI R0611) and loaded to a gel. A 100 bp DNA ladder (MBI SM0241), 6 μl, containing 3 μg DNA was also loaded. The gel was a 2% agarose gel consisting of: TAE buffer (Invitrogen 15558-034), agarose 2%, ethidium bromide, 267 μg/ml. The gel was ran with an electrophoresis power supply E835 (Consort) in a tray HU13 (Jencons) and finally scanned with Flour-S multimager (Biorad) and analysed with the software Quantity One (Biorad).

TABLE 2

Primer pairs used for amplification
of the c-myc-ER construct.

| | | |
|---|---|---|
| Primer pair | Sequence | Product size (bp) |

| | | |
|---|---|---|
| c-myc 1235S/ | 1235S: 5′-CTCCTGGCAAAAGGTCAGAG | 590 |
| M-ER 1131AS | M-ER 1131AS: 5′-AAGGACAAGGCAGGGCTATT | |
| c-myc 1585S/ | c-myc 1585S: 5′-AAAGGCCCCCAAGGTAGTTA | 240 |
| M-ER 1131AS | M-ER 1131AS: 5′-AAGGACAAGGCAGGGCTATT | |

### TaqMan

A standard TaqMan protocol was followed in a 96 well optical reaction plate (AB Applied biosystems). All probes and primers used were from MWG-Biotech AG and all probes were labelled with 3′ TAMRA and 5′ FAM, see table 3. The reaction plate was sealed with optical adhesive cover film (AB Applied biosystems). The TaqMan plate was centrifuged 1 minute at 500 g before loaded to the TaqMan machine (ABI prism 7000 sequence detection system). Finally the result from TaqMan was analysed with ABI prism 7000 SDS software.

TABLE 3
Primers and probes used for TaqMan

| | | |
|---|---|---|
| Target gene | Probe/primers | Sequence |

| | | |
|---|---|---|
| Human | Probe: | 5′-ACATTCAAGCAGATCTCCATGGCAGCA-3′ |
| serum | Forward primer: | 5′-AGTTTGCAGAAGTTTCCAAGTTAGTG-3′ |
| albumin | Reverse primer: | 5′-AGGTCCGCCCTGTCATCAG-3′ |
| Human | Probe: | 5′-CGGTGCCATCCCTTGACTCAACCT-3′ |
| cyp3A4 | Forward primer: | 5′-TCTGGTGTTCTCAGGCACAGA-3′ |
| | Reverse primer: | 5′-CAACCAGAAAAACCCGTTGTTC-3′ |
| Human | Probe: | 5′-CCGTAAGTGGAGCCTGATTTCCCTAAGGA-3′ |
| cyp3A7 | Forward primer: | 5′-AAAGGCTGAGTCAAGGGATGAG-3′ |
| | Reverse primer: | 5′-AGCTTTCTTAAAGAGCAAACCAGAA-3′ |
| GAPDH | Probe: | 5′-ACCACAGTCCATGCCATCACTGCCA-3′ |
| (multi | Forward primer: | 5′-CAAGGTCATCCATGACAACTTTG-3′ |
| spices | Reverse primer: | 5′-GGGCCATCCACAGTCTTCTG-3′ |

### Telomerase Assay

Cells from clone 20, p2 were cultured in T25 flasks for 1 week in media with 0, 100 nM, 1 μM and 10 μM 4-OHT for 1 week. The cells had been in culture for 2 months. Uninfected hepatocytes from the same liver were also used for the assay. These cells had been in culture for 3 weeks. The assay was performed according to the instructions for the Telo TAGGG telomerase PCR ELISAPWs (Roche). The plate reader GENios (Tecan) was used to analyse the results. Some of the fluorescence values were too high for the plate readers' spectra so a change to the protocol was done and the fluorescence was measured from only ⅓ of the final volume.

Results

### Results with Human Primary Foetal Liver Cells

Dissociated human foetal liver and cells were stained after 1 day in culture in 96 well plates for the hepatocyte markers albumin and ck-18. Almost all cells showed positive staining for both albumin and ck-18, indicating that the liver dissociation is good and that most cells in the early cultures are hepatocytes or hepatocyte-like cells. Enrichment of cells expressing the epithelial surface antigen recognised by the HEA-125 antibody was performed before plating the cells and some of the cells were fixed and stained. The HEA-125 positive cells were also positive for albumin. The estimation of the human foetal hepatocyte proliferation rate at different number of days in culture was done with staining against Ki67. The cells positive for Ki67 and total cell numbers was counted for random areas for 4 different days. The result from the Ki67 staining shows that the cells proliferate well during the first week in the arginine-free Williams media but the proliferation rate goes down after longer time, which agrees with the observations that the foetal liver cells did not survive long-term in the arginine-free Williams media. This suggests that the cells proliferated well and the majority of the cells are hepatocytes or hepatocyte like cells when the infection took place. The arginine-free media suppress the proliferation of fibroblast and other nonparenchymal liver cells, allowing for a successful infection of the hepatocytes with the transgene. For the selection of the infected bulk population a titre of neomycin was performed on uninfected human foetal liver cells as described in materials and methods. The cells were exposed to neomycin ranging from 0-900 μg/ml media for 12 days, in triplicate in 6 well plates. Hepatocytes survive the used 250 μg/ml concentration for the 2 week selection. All PCR results for the transgene were positive, the majority of the noninfected cells died during the selection.

### Results for Human Hepatocyte Clones

Clones of human foetal hepatocytes were obtained after the cells had been plated at disperse cell numbers on tissue culture plastic ware and after selection. More then 50 clones were derived. Hepatocytes from early passages were frozen in liquid nitrogen from more then 20 clones. Some frozen vials have been thawed and used in further tissue culture and trypan blue exclusion has showed a viability of 70-95% after freezing/thawing. PCR has confirmed that the c-myc-ER construct has integrated in all clones tested. All clones that have been stained for albumin (19 clones) have shown positive staining. 19 clones have been stained for AFP but no staining has been positive. 8 clones were analysed with TaqMan for expression of cyp3A7 and cyp3A4. The cell pellets from these clones were harvested after 85 days in culture. The results were standardised to human genomic DNA and to the internal housekeeping gene GAPDH. cyp3A7 was still present in all clones after 85 days in culture. c-myc staining showed that c-myc is expressed in a cell line where the integration of the immortalising construct was confirmed with PCR. The telomerase expression assay indicated that culturing the hepatocyte in 0.5-10 μM 4-OHT up regulates telomerase expression and is therefore suitable for immortalisation.

### EXAMPLE 2

### Materials and Methods

All chemicals were purchased from Sigma (Dorset, UK) unless otherwise stated.

### Tissue Culture

Cells were grown at 37° C. in 5% CO₂ in millennium incubators. The medium was changed three times per week and cells were passaged when approximately 90% confluent.

### Media

The following types of media were used in the culture of liver tissue and clones. Medium was sterile filtered with 1 L filter units (Nalgene, N.Y., USA) and pre-warmed to 37° C. before use.

Medium A: Dulbecco's modified eagle's medium (DMEM) (Invitrogen, Paisley, UK), 10% foetal bovine serum (FBS) (Hyclone Perbio Science, UT, USA), gentamycin (50 mg/ml), L-glutamine (2 mM), and epidermal growth factor (EGF) (20 ng/ml).
Medium B: Arginine-free Williams E standard (Invitrogen, Paisley, UK): DMEM (Invitrogen, Paisley, UK) (1:1), 10% FBS, EGF (20 ng/ml).
Medium C: DMEM (+Glutamax) (Invitrogen, Paisley, UK), 10% FBS, 1× non-essential amino acids.
Medium D: DMEM (+Glutamax), 10% FBS, L-glutamine, 2 mM, and penicillin-streptomycin (100 IU/ml) (Invitrogen, Paisley, UK).
Medium E: Arginine-free Williams E standard medium, sodium bicarbonate (2.2 g/l), dialysed FBS (10%) (Gibco 26400-044), L-glutamine (2 mM), Insulin (100 nM), penicillin-streptomycin 100 IU/ml, L-ornithine (0.4 mM), hydrocortisone (5.5 mM), EGF (20 ng/ml).

### Collagen Coating

Liver tissue and clones were maintained on collagen-coated tissue culture plasticware. Collagen solution from calfskin was diluted with sterile water to a final concentration of 8 mg/cm². Following a minimum of 2 h incubation with the collagen solution, flasks were rinsed twice with sterile hanks balanced saline solution (HBSS) (Invitrogen, Paisley, UK).

### Liver Origin and Processing

Local NHS ethical committee approval was obtained to collect human foetal livers following terminated pregnancies.

Liver tissue was received on wet ice in RPMI medium after shipment. The liver was washed twice in 4° C. calcium-free HEPES pH 7.2 (Invitrogen, Paisley, UK) and once in 4° C. calcium-free HEPES pH 7.2 with ethylene glycol-bis(2-aminoethylether)-N,N,N′,N′,-tetraacetic acid (EGTA) (0.5 mM). The liver was minced with scalpels and put into a 37° C. enzyme solution for 20 min consisting of calcium-free HBSS containing collagenase A (0.05%) (Roche, East Sussex, UK), DNase I (0.005%) (Roche, East Sussex, UK), hylaronidase (0.0125%), and dispase II (0.025%) (Roche, East Sussex, UK), supplemented with 5 mM calcium chloride. The dissociated liver was filtered through a sterile nylon mesh and collected in 4° C. medium A. To remove some of the small blood cells and other cells, the cell suspension was centrifuged three times at 50 g and the supernatant discarded each time. A cell number and viability count was performed with a haemocytometer. The cells were then plated on the pre-coated tissue culture plasticware in medium A and left to attach for 4 h at 37° C. Following this the cells were washed once with 37° C. phosphate buffered saline (PBS) (Invitrogen, Paisley, UK) to remove debris and unattached cells. Medium B was then added to the cells.

### Passaging

Cells were rinsed once with 37° C. PBS and to detach cells from the plastic lx trypsin-versine (BioWhittaker, ME, USA) was added for up to 10 min. An equal volume of medium was applied to neutralise the trypsin, then the cell suspension was transferred to a falcon tube for centrifuging at 500 g for 5 min. The supernatant was removed and cells resuspended in medium and a cell count performed using a haemocytometer. Cells were seeded into collagen-coated plasticware at a density of 8,000 cells per cm².

### Freezing and Thawing Cells

To freeze cells the cell pellet was resuspended in a mixture of 900 ml of medium and 100 ml of cryosure-dimethyl sulfoxide (DMSO) (Quest Biomedical, West Midlands, UK) in a cryo-safe vial (Nunc, N.Y., USA). The cells were placed at −80° C. for 24 h in a “Mister Frosty” cryopreservation freezing container (Nalgene, N.Y., USA), where the temperature decreases at approximately 5° C. per min. After 24 h the vials were placed in liquid nitrogen for long-term storage.

To thaw cells, the selected vial was removed from liquid nitrogen and placed in a 37° C. water bath until thawed. The cell suspension was mixed with 10 ml medium and a pellet recovered by centrifugation at 500 g for 5 min. The pellet was resuspended in fresh medium, a cell number and viability count performed and the cells seeded onto collagen-coated plasticware.

### Harvesting of Virus

Virus producer cells from clone Fly-C042 were cultured to confluence in 175 cm² flasks using medium D. Once confluent the flasks were rinsed three times with PBS before medium E (18 ml per 175 cm² flask) was added to the flasks and incubated for 8 h. The medium was harvested and filtered using a 0.45 mm filter (Sartorius, Surrey, UK), split into 5 ml aliquots and snap frozen in liquid nitrogen for storage at −80° C.

### Infection and Selection

Cells were seeded at concentrations ranging from 200 cells/dish to 100% confluent in 10 cm dishes. Three days after the dissociation of the human foetal liver, the cells were washed once with PBS and exposed to 5 ml of the virus containing medium, plus hexadimethrine bromide (8 mg/ml), for 8 h. After this incubation period medium B was added overnight and the infection repeated the next day. Following infection 4-hydroxytamoxifen (OHT) was added to all media at a concentration of 0.1-1.0 mM.

Two days after infection the cells were passaged into 15 cm dishes, and seeded with 1,000 cells per dish in preparation for selection 48 h later. Cells successfully infected and expressing the c-mycER construct are resistant to neomycin (also known as geneticin and G418). A titre of neomycin was performed to determine the correct concentration to use so that non-infected cells were eliminated while infected cells were not. Based on results of the titre, cells were selected by 2 weeks exposure to 250 mg/ml neomycin in medium B.

### Cyquant Proliferation Assay

Growth rate was assessed using the Cyquant cell proliferation assay (Molecular Probes, Paisley, UK). Cells were grown in multiwell plates or 25 cm² flasks and experiments were performed according to the manufacturers instructions.

### Immunocytochemistry

Immunocytochemistry was used for early assessment of the cell line. This method does not allow quantification, but provides useful information on the presence of liver markers. The human hepatoma cell line HuH7 was used as a positive control for liver markers, while a myc-immortalised neural stem cell line (197VM) cells were used as a negative control.

All staining was performed in multiwell plates using the following method. Medium was aspirated and cells washed once with PBS prior to fixing using 4% paraformaldehyde for 15 min at room temperature. Cells were then washed three times in PBS, before being permeabilised for 15 min using 0.1% triton x-100 in PBS. Cells were washed with PBS once more then blocked for 1 h in 10% normal goat serum (NGS) (Vector Laboratories, CA, USA) in PBS. Primary antibodies were applied in 1% NGS/PBS for 1 h at room temperature. Cells were washed three times in PBS then exposed to the secondary antibody in 1% NGS/PBS for 1 h at room temperature. Cells were washed three times in PBS and Hoechst, a dye that stains DNA, was added for 2 min at room temperature (1:25,000 in PBS). Cells were washed a further three times in PBS and left in the final wash. Analysis was performed using a fluorescence microscope (Leica DMRA). The secondary antibody excited at a wavelength of ˜488 nm and gave emission at ˜525 nm (green emission) while Hoechst excited at ˜350 nm and emitted at ˜425 nm (blue emission).

### TaqMan

A standard TaqMan protocol was following using a 96 well optical reaction plate (Applied Biosystems, CA, USA). All probes and primers were supplied by MWG-Biotech, Ebersberg, Germany (see table 3). All probes were labelled with 3′TAMRA and 5′FAM. The reaction plate was sealed with optical adhesive cover (Applied Biosystems, CA, USA) and centrifuged for 1 min at 500 g before loading onto the TaqMan machine (ABI prism 7000 sequence detection system) and results analysed with ABI prism 7000 SDS software.

TABLE 4
Primers and probes used for TaqMan

| Target | | |
|---|---|---|
| Gene | Probe/primers | Sequence |
| Human | Probe: | 5′-CGG TGC CAT CCC TTG ACT CAA CCT-3′ |
| CYP3A4 | Forward primer: | 5′-TCT GGT GTT CTC AGG CAC AGA-3′ |
| | Reverse Primer: | 5′-CAA CCA GAA AAA CCC GTT GTT C-3′ |
| Human | Probe: | 5′-CCG TAA GTG GAG CCT GAT TTC CCT AAG GA-3′ |
| CYP3A7 | Forward primer: | 5′-AAA GGC TGA GTC AAG GGA TGA G-3′ |
| | Reverse Primer: | 5′-AGC TTT CTT AAA GAG CAA ACC AGA A-3′ |
| b-Actin | Probe: | 5′-CAT CAC TAT CGG CAA TGA GCG GTT CC-3′ |
| | Forward primer: | 5′-GAG CTA TGA GCT GCC TGA C-3′ |
| | Reverse Primer: | 5′-AGT TTC ATG GAT GCC ACA GGA-3′ |

### Results

### Dissociation, Infection and Clone Selection

Tissue was received at 15 weeks gestation, dissociated and infected as described. Following 2 weeks selection, individual clones were picked and expanded. The clone designated LIV0A07 showed promising characteristics and was subjected to further characterisation. It has been maintained in long-term culture (beyond 30 passages) without senescence.

Tissue was maintained in medium B supplemented with 0.1 mM 4-OHT through selection and early clone growth, and then LIV0A07 was placed in medium C supplemented with 1.0 mM 4-OHT after clone passage 3.

### Cyquant Proliferation Assay

Growth rate was assessed using the Cyquant cell proliferation assay to compare growth of LIV0A07 over 5 days in 0-1.0 mM 4-OHT.

Little proliferation was observed in cells grown in 0 and 0.1 mM 4-OHT. Over the five days of the assay, cells grown in the presence of 0.5 and 1.0 mM 4-OHT expanded to a greater extent, however 1.0 mM 4-OHT may be slightly toxic to the cells (highlighted by initial slow proliferation after seeding). Hence, LIV0A07 shows 4-OHT dependent growth with an optimum concentration of 0.5-1.0 mM 4-OHT in the medium.

### Immunocytochemistry

The positive control cell line, HuH7, was positive for albumin and cytokeratin-18 (CK-18), while 197VM cells were negative for these markers In cells containing the c-mycER construct addition of 4-OHT promotes proliferation, while removal of 4-OHT promotes differentiation of the cell to display the mature phenotype. LIV0A07 showed positive staining for the hepatocyte markers albumin, and CK-18. Staining appeared to be slightly stronger in differentiated cultures (grown in the absence of 4-OHT for 48 h).

No positive staining was observed when using an anti-fibroblast antibody for any of the cell types. This antibody cross-reacts with the b-subunit of prolyl-4-hydrolase and with disulphideisomerase.

### PCR for c-mvcER

PCR performed using the primer pair c-myc 1585S, and M-ER 1131AS confirmed expression of the c-mycER construct in LIV0A07.

### TaqMan

Analysis of LIV0A07 mRNA using TaqMan demonstrated expression of the cytochrome P450 3A4 and 3A7 enzymes, but under these conditions at a lower level than that seen in the control cell line, HuH7. HuH7 levels were approximately 10 fold higher than the maximum seen for LIV0A07.

A specific increase in CYP3A4 mRNA expression was observed in differentiated cultures of LIV0A07 (grown in the absence of 4-OHT for 72 h). Levels were approximately 15 fold higher in differentiated LIV0A07 compared to undifferentiated cultures, while no effect of 4-OHT was observed in samples from HuH7 cells.

CYP3A7 levels did not display the same differentiation induced increase as CYP3A4 in LIV0A07 samples, but levels of this enzyme did increase with time after seeding. Lowest levels were detected at 72 h and highest at 1 week post seeding. The results show that the cells of the invention have increased cytoP450 levels and therefore more approximate regular hepatocytes. This example demonstrates that clonal, conditionally-immortal, cell lines can be developed from human hepatocytes that express hepatocyte functional markers that are maintained over multiple cell generations (beyond 30 passages). Other cell lines with different phenotypic profiles have been developed.

### REFERENCES

1. Razvi E. The Landscape of the Opportunity, Technologies, and Products Dominating the Space. Issue of Drug & Market Development 2003.
2. Gomez-Lechon M J, Donato M T, Castell J V, Jover R. Human hepatocytes as a tool for studying toxicity and drug metabolism. Curr. Drug Metab 2003; 4:292-312.
3. Oesterheld J R. A review of developmental aspects of cytochrome P450. J. Child Adolesc. Psychopharmacol. 1998; 8:161-174.
4. Rambhatla L, Chiu C P, Kundu P, Peng Y, Carpenter M K. Generation of hepatocyte-like cells from human embryonic stem cells. Cell Transplant. 2003; 12: 1-11.
5. Kono Y, Yang S, Letarte M, Roberts E A. Establishment of a human hepatocyte line derived from primary culture in a collagen gel sandwich culture system. Exp. Cell Res. 1995; 221: 478-485.
6. Fukaya K, Asahi S, Nagamori S et al. Establishment of a human hepatocyte line (OUMS-29) having CYP 1A1 and 1A2 activities from fetal liver tissue by transfection of SV40 LT. In Vitro Cell Dev. Biol. Anim 2001; 37: 266-269.
7. Rodriguez-Antona C, Donato M T, Boobis A et al. Cytochrome P450 expression in human hepatocytes and hepatoma cell lines: molecular mechanisms that determine lower expression in cultured cells. Xenobiotica 2002; 32: 505-520.
8. Hanada S, Kayano H, Jiang J et al. Enhanced in vitro maturation of subcultivated fetal human hepatocytes in three dimensional culture using poly-L-lactic acid scaffolds in the presence of oncostatin M. Int. J. Artif. Organs 2003; 26: 943-951.
9. Yuan J, Liu L, Shimada M et al. Induction, expression and maintenance of cytochrome P450 isoforms in long-term cultures of primary human hepatocytes. ALTEX. 2004; 21 Suppl 3: 3-11.
10. Hamilton G A, Jolley S L, Gilbert D, Coon D J, Barros S, LeCluyse E L. Regulation of cell morphology and cytochrome P450 expression in human hepatocytes by extracellular matrix and cell-cell interactions. Cell Tissue Res. 2001; 306: 85-99.
11. Kim B H, Sung S R, Choi E H et al. Dedifferentiation of conditionally immortalized hepatocytes with long-term in vitro passage. Exp. Mol. Med. 2000; 32: 29-37.
12. Jacob J R, Estlack L E, Lanford R E. Immortalization of chimpanzee hepatocytes with an amphoteric retrovirus encoding simian virus 40 T antigen. Exp. Cell Res. 1992; 200: 205-210.
13. Wang J, Lu X X, Chen D Z, Li S F, Zhang L S. Herpes simplex virus thymidine kinase and ganciclovir suicide gene therapy for human pancreatic cancer. World J. Gastroenterol. 2004; 10: 400-403.
14. Key G, Kubbutat M H, Gerdes J. Assessment of cell proliferation by means of an enzyme-linked immunosorbent assay based on the detection of the Ki67 protein. J. Immunol. Methods 1994; 177:113-117.
15. Davis H E, Morgan J R, Yarmush M L. Polybrene increases retrovirus gene transfer efficiency by enhancing receptor-independent virus adsorption on target cell membranes. Biophys. Chem. 2002; 97:159-172.
16. Wege H, Le H T, Chui M S et al. Telomerase reconstitution immortalizes human fetal hepatocytes without disrupting their differentiation potential. Gastroenterology 2003; 124: 432-444.
17. Singh D, Rigby P W. The use of histone as a facilitator to improve the efficiency of retroviral gene transfer. Nucleic Acids Res. 1996; 24: 3113-3114.
18. Littlewood T D, Hancock D C, Danielian P S, Parker M G, Evan G I. A modified oestrogen receptor ligand-binding domain as an improved switch for the regulation of heterologous proteins. Nucleic Acids Res. 1995; 23: 1686-1690.
19. Crosby H A, Hubscher S G, Joplin R E, Kelly D A, Strain A J. Immunolocalization of OV-6, a putative progenitor cell marker in human fetal and diseased pediatric liver. Hepatology 1998; 28: 980-985.
20. Waring, J. F., et al., Clustering of hepatotoxins based on mechanism of toxicity using gene expression profiles. Toxicol. Appl. Pharmacol., 2001. 175(1): p. 28-42.
21. Waring, J. F., et al., Microarray analysis of hepatotoxins in vitro reveals a correlation between gene expression profiles and mechanisms of toxicity. Toxicol. Lett., 2001. 120(1-3): p. 359-68.
22. Barton, M. C. and D. N. Stivers, Microarray analysis of hepatic-regulated gene expression: specific applications and non-specific problems. Hepatology, 2002. 35(3): p. 727-9.
23. Hamadeh, H. K., et al., Gene expression analysis reveals chemical-specific profiles. Toxicol. Sci., 2002. 67(2): p. 219-31.
24. de Longueville, F., et al., Use of a Low-Density Microarray for Studying Gene Expression Patterns Induced by Hepatotoxicants on Primary Cultures of Rat Hepatocytes. Toxicol. Sci., 2003. 75(2): p. 378-392.
25. Adam, B. L. et al., Proteomic approaches to biomarker discovery in prostate and bladder cancers. Proteomics, 2001. 1: p. 1264-1270.
26. Seow, T. K, et al., Hepatocellular carcinoma: From bedside to proteomics. Proteomics, 2001. 1: p. 1249-1263.
27. Anderson, N. L., A two-dimensional gel database of rat liver proteins useful in gene regulation and drug effect studies. Electrophoresis, 1991. 12: p. 907-930.
28. Richert et a., Toxicology in vitro, 2002; 16: 89-99.

We claim:

1. A mammalian hepatocyte comprising, as a single polypeptide, a fusion protein comprising an oncoprotein of the myc family and an estrogen receptor, or functional fragments thereof.

2. The hepatocyte according to claim 1, wherein the oncoprotein is c-myc.

3. The hepatocyte according to claim 1, in culture media.

4. The hepatocyte according to claim 1, wherein the hepatocyte is a human hepatocyte.

5. The hepatocyte according to claim 1, comprising a polynucleotide encoding the fusion protein.

6. The hepatocyte according to claim 5, wherein the polynucleotide is integrated into the genome of the hepatocyte.

7. The hepatocyte according to claim 1, wherein the fusion protein comprises a mouse receptor and a human c-myc protein.

8. The hepatocyte according to claim 1, wherein the estrogen receptor contains a mutation that prevents high affinity binding to 17β-oestradiol.

9. The hepatocyte according to claim 1, comprising the polynucleotide sequence identified herein as SEQ ID NO: 1.

10. The hepatocyte according to claim 1, wherein the hepatocyte expresses markers characteristic of hepatocytes in vivo.

11. The hepatocyte according to claim 10, wherein the markers include members of the cytochrome P450 family, albumin, α-fetoprotein and cytokeratin-18.

12. The hepatocyte according to claim 1, wherein the hepatocyte is conditionally immortalized when contacted with a ligand of the estrogen receptor.

13. The hepatocyte according to claim 12, wherein the ligand is 4-hydroxytamoxifen.

14. A method of conditionally immortalizing a hepatocyte, comprising:
(i) expressing in the hepatocyte a fusion protein comprising an oncoprotein of the myc family and an estrogen receptor, or functional fragments thereof, expressed as a single polypeptide chain; and
(ii) contacting the hepatocyte formed by step (i) with a ligand of the estrogen receptor, thereby conditionally immortalizing the hepatocyte.

15. A method of evaluating the suitability of a compound for use as a drug, in vitro, comprising:
(i) contacting a hepatocyte with the potential drug compound, wherein the hepatocyte comprises, as a single polypeptide, a fusion protein comprising an oncoprotein of the myc family and an estrogen receptor, or functional fragments thereof; and
(ii) measuring the response of the hepatocyte, to thereby determine the effect of the compound on the hepatocyte and evaluate the suitability of the compound for use as a drug.

16. A method for treating a disorder, comprising administering an effective amount of hepatocytes to a subject in need thereof, wherein the hepatocytes comprise, as a single polypeptide, a fusion protein comprising an oncoprotein of the myc family and an estrogen receptor, or functional fragments thereof.

17. The method of claim 16, wherein the disorder is a disorder of the liver.

18. The method of claim 17, wherein the disorder of the liver is selected from the group consisting of cancer, cirrhosis, and hepatitis.

19. A liver assisting device comprising at least one hepatocyte comprising, as a single polypeptide, a fusion protein comprising an oncoprotein of the myc family and an estrogen receptor, or functional fragments thereof.
